(19) United States
(12) Patent Application Publication
(10) Pub. No.: US 2009/0187392 A1
(43) Pub. Date: Jul. 23, 2009

(54) SYSTEM AND METHOD FOR MONITORING A HEALTH CONDITION OF AN ANIMAL

(75) Inventors: Frank Riskey, Boise, ID (US); Brian Douglas Griebe, Star, ID (US); Cathy Ann King, Boise, ID (US); Layne Ellen Simmons, Middleton, ID (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: TENXSYS INC., Eagle, ID (US)

(21) Appl. No.: 12/018,546

(22) Filed: Jan. 23, 2008

(51) Int. Cl. G06G 7/60

(52) U.S. Cl. 703/11

(57) ABSTRACT

An ingestible bolus may be disposed within the stomach of an animal. The bolus may comprise one or more sensors to monitor one or more internal and/or external animal characteristics. The bolus may comprise a data transmitter in wireless communication with a base station. The base station may receive messages from the bolus comprising one or more measured animal characteristics. The base station may comprise a process to monitor the animal and/or be in communication with a process to monitor the animal. The process may build an animal profile based upon the observed characteristics of the animal, the characteristics of other animals in a group associated with the animal, and/or characteristics associated with the breed and/or sex of the animal. Based on this profile, the process may detect a change in a health condition of the animal. Such heath conditions may include, but are not limited to, an estrus condition in the animal, off-feed condition, a nominal condition, the animal leaving an enclosure, or the like.

### TECHNICAL FIELD

This disclosure relates generally to systems and methods for monitoring a health condition of an animal and, in particular, to systems and methods for monitoring one or more animal characteristics to detect a health condition of the animal.

### BRIEF DESCRIPTION OF THE DRAWINGS

The various aspects and advantages of the invention are described by way of example in the following description of several embodiments and attached drawings. It should be understood that the accompanying drawings depict only typical embodiments and, as such, should not to be considered to limit the scope of the claims. The embodiments will be described and explained with specificity and detail in reference to the accompanying drawings in which:

FIG. 1 is a block diagram of one embodiment of an animal monitoring system;

FIG. 2 is a block diagram of one embodiment of a bolus comprising one or more sensors;

FIG. 3 is a block diagram of an animal information data structure;

FIG. 4 is a block diagram of an animal monitoring system, comprising an animal monitoring service in communication with a base station and bolus disposed within a stomach of a ruminant animal;

FIG. 5 is a flow diagram of one embodiment of a method for establishing and updating an animal characteristic model of an animal health-condition pattern;

FIG. 6 is a flow diagram depicting one embodiment of a method for monitoring a health condition of an animal;

FIG. 7 is a flow diagram depicting one embodiment of a method for determining a probability that an animal has/is in a particular health condition; and

FIG. 8 is a flow diagram depicting one embodiment of a method for detecting a health condition of an animal.

### DETAILED DESCRIPTION OF PREFERRED EMBODIMENTS

This disclosure relates generally to systems and methods for monitoring a health condition of an animal and, in particular, to systems and methods for monitoring one or more animal characteristics to detect a health condition of the animal.

It will be readily understood that the components of the present invention, as generally described and illustrated in the Figures herein, could be arranged and designed in a wide variety of different configurations. Thus, the following detailed description of the embodiments of the apparatus, system, and method of the disclosure is not intended to limit the scope of the disclosure, as claimed, but is merely representative of possible embodiments of the disclosure.

In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the Figures herein could be arranged and designed in a wide variety of different configurations.

The order of the steps or actions of the methods described in connection with the embodiments disclosed may be changed as would be apparent to those skilled in the art. Thus, any order in the Figures or description is for illustrative purposes only and is not meant to imply a required order, unless specified to require an order.

Certain aspects of the embodiments described may be illustrated as hardware components, or software modules or components. As used herein, a software module or component may include any type of computer instruction or computer executable code located within a memory device and/or transmitted as electronic signals over a system bus or wired or wireless network. A software module may, for instance, comprise one or more physical or logical blocks of computer instructions, which may be organized as a routine, program, object, component, data structure, etc. that performs one or more tasks or implements particular abstract data types. In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

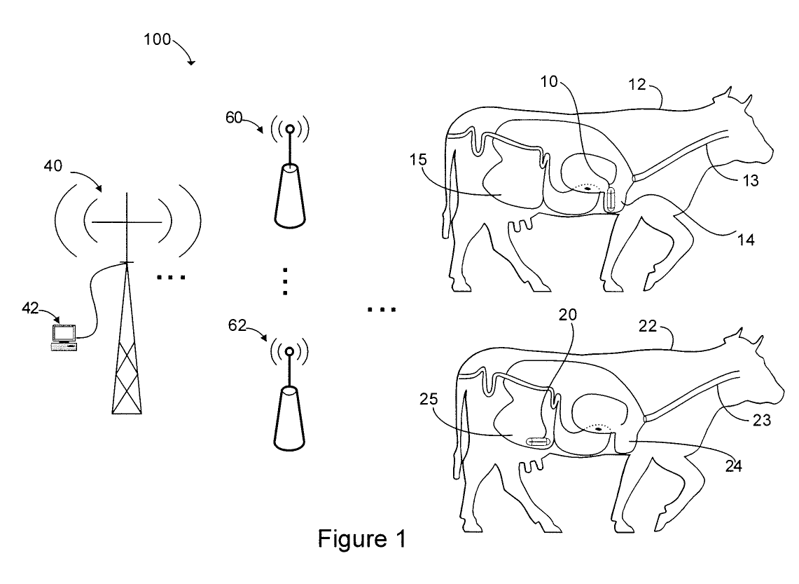

Turning now to FIG. 1, a block diagram 100 of one embodiment of a system for monitoring a health condition of an animal is depicted. In FIG. 1, an ingestible bolus 10, 20 may be disposed within the body of an animal 12, 22. Bolus 10, 20 may be configured to be ingested via the esophagus 13, 23 of a ruminant animal 12, 22, such as a bovine. In this embodiment, bolus 10, 20 may be configured to have a size and density which will enable it to remain within the stomach of a bovine 12, 22, ensuring that it is not regurgitated from the animal's reticulum 14, 24 and/or rumen 15, 25.

FIG. 1 shows ingestible bolus 10 within reticulum 14 of ruminant animal 12 and ingestible bolus 20 within rumen 24 of ruminant animal 22. Ingestible bolus 10, 20 may be maintained in any stomach and/or stomach chamber capable of holding bolus 10, 20. Bolus 10, 20 may be capable of remaining in the animal's reticulum 14, 24 and/or rumen 15, 25 throughout the life of the animal 12, 22.

In an alternative embodiment, bolus 10, 20 may be injected into and/or implanted under the skin of an animal 12, 22 or otherwise implanted within the body of an animal 12, 22.

Bolus 10, 20 may comprise wireless communications means to allow bolus 10 to be in wireless communication with base station 40. Base station 40 may comprise a computing device 42 communicatively coupled to base station 40. Computing device may be any general and/or specific purpose computing device known in the art.

The base station 40 and/or computing device 42 may be monitored by and/or in communication with an animal manager. As used herein, an animal manager may refer to any person, machine, and/or used to manage one or more animals. The animal manager may be in charge of and/or responsible for one or more animals monitored by the systems and methods disclosed herein. An animal manager may comprise one or more automated systems such as feeding, heating, cooling and other systems. An animal manager may further comprise a human animal manager and/or veterinarian. Base station 40 and/or computing system 42 may be configured by and/or interact with the animal manager to monitor and/or respond to changes in animal 12, 22 health condition.

The wireless communication means of bolus 10, 20 may comprise a wireless transmitter and/or receiver operating at 900 MHz or some other suitable radio frequency (RF). In some embodiments, such wireless communication may be two-way, allowing bolus 10, 20 to transmit and receive data from base station 40. In other embodiments, bolus 10, 20 may only be capable of transmitting data to base station 40.

Animals 12 and 22 may be capable of roaming over a relatively large area, such as a feed lot, dairy, range area, or the like. As such, the distance between base station 40 and bolus 10, 20 may become greater than the wireless communication range of bolus 10, 20. In this case, one or more wireless transponders 60, 62 may be deployed to increase the communications range of bolus 10, 20 to base station 40. Transponder(s) 60, 62 may receive wireless transmissions from bolus 10 and retransmit them at a higher power and/or a different frequency to allow such transmissions to be received by base station 40. Similarly, in this embodiment, transponder(s) 60, 62 may receive transmissions from base station 40 to bolus 10 and retransmit them at higher power so that they may be received by bolus 10, 20.

Multiple wireless transponders 60, 62 may be disposed within the vicinity of animals 12, 22. For example, FIG. 1 depicts two wireless transponders 60, 62 that may be in wireless communication with bolus 10, 20. Where there are multiple base stations 40 and/or transponders 60, 62 within the wireless communication range of bolus 10, 20, boluses 10,20 may be configured to communicate with only the base station 40 and/or transponder 60, 62 with the strongest communications signal. In this embodiment, each base station 40 and transponder 60, 62 may be configured to communicate on a separate frequency and/or channel within an RF frequency range (e.g., 900 MHz). The bolus 10, 20 may be configured to receive data on each of the communications channels used by base station 40 and transponders 60, 62 to listen for the strongest signal received on each. Upon determining the best (e.g., strongest) wireless communication channel (e.g., transponder 60, 62 and/or base station 40), bolus 10, 20 may transmit and/or receive data using only that channel. Bolus 10, 20 may be configured to periodically re-evaluate the available channels to allow bolus 10, 20 to adapt to changes in animal position and/or changes in base station 40 and/or transponder 60, 62 signal strength and/or configuration. Similarly, if bolus 10, 20 is out of communication range with any of transponder(s) 60, 62 and base station 40, bolus 10, 20 may be configured to cease transmission and/or only transmit transponder 60, 62 and/or base station 40 discovery messages.

Although FIG. 1 depicts two boluses 10 and 20 and two transponders 60 and 62, one skilled in the art would recognize that an animal monitoring system according to the teachings of this disclosure could comprise a virtually unlimited number of transponders 60, 62 animals 12, 22, and/or boluses 10, 20. As such, this disclosure should not be read as limited to any particular transponder 60, 62, animal 12, 22, and/or bolus 10, 20 configurations.

Bolus 10, 20 may comprise one or more sensors to detect one or more characteristics of animal 12, 22. In this embodiment, bolus 10, 20 may wirelessly transmit data corresponding to monitored animal characteristics to base station 40. Monitored animal characteristics may include physiological characteristics, such as animal temperature, stomach pH, blood pH, heart rate, respiration, stomach or rumen contractions, and the like. Monitored animal characteristics may also include non-physiological characteristics, such as animal movement and/or motion activity, animal location, and the like.

The wireless communication characteristics of bolus 10, 20 between base station 40 and/or transponder(s) 60, 62 may allow base station 40 to determine location information relating to animal 12, 22. In an embodiment employing only a single base station 40, base station 40 may be configured to determine the animal's 12, 22 distance from base station 40. This may be done in a variety of ways including, but not limited to: determining distance based upon wireless signal strength; determining distance from timestamp information in the wireless message; or the like.

As shown in FIG. 1, the system may comprise more than one base station 40 and/or transponder(s) 60, 62. In this case, base station 40 may determine location information relating to animal 12, 22 using well-known wireless communications triangulation methods.

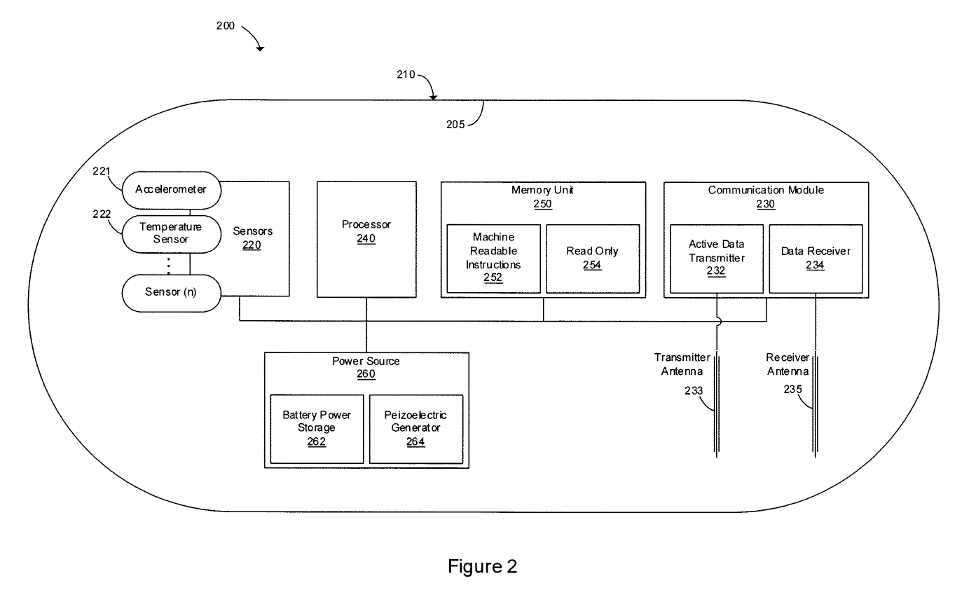

Turning now to FIG. 2, a block diagram of one embodiment 200 of a bolus 210 is depicted. The components of bolus 210 may be disposed within an enclosure 205. Enclosure 205 may be formed from any material capable of remaining within the stomach of an animal without deteriorating and/or degrading. In addition, enclosure 205 may be formed of a material unlikely to produce an adverse reaction in an animal when implanted therein (e.g., within the rumen or reticulum of the animal). In one embodiment, enclosure 205 may be formed from a non-toxic plastic material.

Bolus 210 may comprise one or more sensors 220 configured to measure one or more animal characteristics. One or more of sensors 220 may detect animal movement and/or motion activity characteristics including, but not limited to: distance traveled by the animal; animal movement frequency; animal movement speed; and the like. In one embodiment, an accelerometer 221 may be used to detect such movement and/or motion activity characteristics. In this embodiment, accelerometer 221 may be a three (3) axis accelerometer capable of detecting animal movement and/or motion activity in each of the Cartesian “x,” “y,” and “z” axes. Bolus 210 may change its orientation while within an animal (e.g., within an animal's rumen and/or reticulum). As such, detection of movement and/or motion activity in only one or two axes may yield inaccurate results.

An acceleration vector magnitude (VM) value may be calculated from the readings of the three axis accelerometer by calculating the square root of the sum of the squares of each of the “x,” “y,” and “z” coordinate axes as shown in Equation 1.1:

VM=\sqrt{x²+y²+z²} Eq. 1.1

A derivative of the vector magnitude (VM) may be approximated by calculating the absolute value of the difference between subsequent vector magnitude values as illustrated in equation 1.2:

$$\frac{\partial {VM}_{n}}{\partial t} = \vert {VM}_{n} - {VM}_{n - 1} \vert \qquad Eq . 1.2$$

The derivative of acceleration calculated per equation 1.2 may be useful in monitoring animal characteristics as it may remove errors caused by “float” movement of bolus 210 within the stomach of the animal or other constant acceleration forces acting on the animal (e.g., gravity). Accordingly, the derivative of the movement vector magnitude may provide an accurate representation of the actual movement and/or motion activity characteristics of the animal.

In addition to detecting movement and/or motion activity, accelerometer 221 may be configured to detect stomach contractions. Since bolus 210 may be disposed in the stomach of an animal (e.g., in a ruminant animal's rumen or reticulum), bolus 210 may be movably affected by animal stomach contractions. Such movement and/or motion activity may be detected by accelerometer 221 as a periodic acceleration. For example, a typical ruminant animal may experience a three (3) stomach contractions every minute while “on-feed” (i.e., every 20 seconds). As used herein, “on-feed” may refer to an animal health condition wherein the animal is actively feeding, and “off-feed” may refer to an animal health condition wherein the animal is no longer and/or intermittently feeding.

In one embodiment, bolus 210 may comprise one or more sensors 220 capable of determining the position of bolus 210, such as a Global Positioning System (GPS) receiver or the like. In this embodiment, a GPS receiver may be used to detect both animal position and animal movement and/or motion activity characteristics.

One or more sensors 220 of bolus 210 may be used to detect physiological characteristics of an animal including, but not limited to: body temperature; heart rate; respiration; stomach contractions; stomach pH; blood pH; and the like. Any number of sensors 220 may be used to detect such characteristics. For example, to detect animal temperature, a temperature sensor 222 may be employed. In this embodiment, temperature sensor 222 may comprise a thermistor, thermocouples or a platinum resistance thermometer, or the like.

It would be understood by one skilled in the sensor arts that any number of sensors 220 could be included within bolus 210 under the teachings of this disclosure. As such, this disclosure should not be construed as limited to any particular sensors 220.

In one embodiment, bolus 210 may comprise a communications unit 230. Communications unit 230 may comprise active data transmitter 232 and data receiver 234. Active data transmitter 232 may be communicatively coupled to transmitter antenna 233. Transmitter antenna 233 may be disposed within enclosure 205 of bolus 210, upon the surface thereof, or may be disposed externally to enclosure 205 of bolus 210. Data receiver 234 may be communicatively coupled to receiver antenna 235. Receiver antenna 235 may be disposed within enclosure 210 of bolus 10, upon the surface thereof, or may be disposed externally to enclosure 210 of bolus 10. In one embodiment, transmitter antenna 233 may be capable of transmitting data at 900 MHz, and receiver antenna 235 may be capable of receiving data at 900 MHz. In another embodiment, transmitter antenna 233 and receiver antenna 235 may be comprised of a single antenna (not shown) used for both data transmission and reception.

Bolus 210 may comprise a processor 240 communicatively coupled to a memory unit 250. In one embodiment, memory unit 250 may comprise machine readable instructions 252 stored thereon. In this embodiment, processor 240 may read and execute machine readable instructions 252 stored on memory unit 250.

Processor 240 may be communicatively coupled to each of sensors 220. Machine readable instructions 252 stored on memory unit 250 may specify a sensor sampling frequency for each of the sensors 220. As used herein, a sensor sampling frequency may determine how often a sensor reading is obtained from a particular sensor 220. For example, a sensor sampling frequency may define how often temperature sensor 222 obtains a temperature sensor reading or sensor sample from the animal. The processor 240 may configure one or more of sensors 220 with a sensor sampling frequency specified by machine readable instructions 252. Alternatively, one or more sensors 220 may be communicatively coupled to memory unit 250 and may be configured to read a sensor sampling frequency directly from the machine readable instructions 252.

Machine readable instructions 252 may specify a sensor reading duration for each of sensors 220. As used herein, a sensor reading duration may define the length of time a particular sensor 220 may obtain a reading. For example, a reading duration may define how long accelerometer 221 reads animal movement and/or motion activity characteristics. A reading duration may specify that accelerometer 221 should read animal movement and/or motion activity characteristics for one minute each time a sensor sample is taken. Processor 240 may configure one or more of sensors 220 with a sensor reading duration specified by machine readable instructions 252. Alternatively, one or more sensors 220 may be communicatively coupled to memory unit 250 and may be configured to read their sensor reading duration directly from machine readable instructions 252.

Machine readable instructions 252 may specify calibration information for one or more sensors 220. In this embodiment, one or more sensors 220 may be tested to determine whether accurate readings are being returned. In the event a particular sensor 220 is not returning accurate readings, calibration data may be stored within memory unit 250 to rectify the readings to a correct value. In this embodiment, sensor 220 may be communicatively coupled to memory unit 250 to allow a sensor 220 to read the calibration data therefrom. Sensor 220 may itself comprise a memory storage location whereon such calibration information may be stored. Machine readable instructions 252 may instruct processor 240 to transfer sensor calibration data stored within memory unit 250 to the memory storage location of a particular sensor 220. In another embodiment, sensor 220 may not comprise a memory storage location and may not be capable of reading memory unit 250. As such, machine readable instructions 252 may configure processor 240 to apply calibration data stored within memory unit 250 to readings returned by sensors 220.

Machine readable instructions 252 may specify that one or more sensors 220 should be deactivated in order to reduce the power consumed by bolus 210. Processor 240 may be communicatively coupled to sensors 220 and may be capable of configuring and/or controlling one or more of sensors 220. Machine readable instructions 252 may specify that one or more sensors 220 should be re-activated.

Processor 240 may be communicatively coupled to sensors 220 and may control the operation and configuration of sensors 220. Processor 240 may poll one or more of sensors 220 at a polling interval (i.e., polling frequency) specified by machine readable instructions 252 stored in memory unit 250. As used herein, polling a sensor refers to obtaining measurement data from one or more sensor 220. Polling a sensor may comprise processor 240 sending a query to a sensor 220, and responsive to this query, sensor 220 may obtain and return to processor 240 the sensor reading. For example, temperature sensor 222 may respond to polling by reading and returning the current animal temperature. In another embodiment, polling a sensor may comprise causing processor 240 to read the current sensor value from a sensor 220. In another embodiment, one or more sensors 220 may be configured to store sensor measurements on memory unit 250. One or more sensors 220 may be configured with a sensor sampling frequency that is greater than the polling frequency of processor 240. As such, sensors 220 may store multiple sensor samplings on memory unit 250 between polling intervals of processor 240. Accordingly, polling a sensor 220 may comprise processor 240 reading all of the sensor readings stored on memory unit 250 for each of the one or more sensors 220.

In another embodiment, sensor 220 may alternatively comprise a memory storage location to store sensor samples. In this embodiment, processor 240 may poll sensor 220 by reading a sensor 220 storage location. In another embodiment, sensor 220 may have a sensor reading duration to allow sensor 220 to measure animal characteristics over time (e.g., an accelerometer sensor 221). Sensor 220 may store such measurements on an internal sensor storage location or on memory unit 250. The processor 240 may poll such a sensor by reading memory 250 or the internal storage location of the sensor 220. In this embodiment, processor 240 may be configured to pre-processes the measurement data before transmission. Such preprocessing may comprise calculating a measurement characteristics, such as mean, standard deviation, etc., compressing the data, and the like. As such, the preprocessing may reduce the amount of data transmitted to the base station (not shown) and thereby reduce power and RF transmission requirements.

In addition to transmitting animal characteristics obtained by one or more bolus 210 sensors 220, bolus 210 may be configured to transmit bolus 210 status information including, but not limited to: power available in power source 260; status of sensors 220, processor 240 and/or memory unit 250, or the like. A base station (not shown) may use this status information to alert an animal manager to a possible problem with the bolus 210 (e.g., power source 260 about to expire, etc.).

It should be understood that bolus 210 may comprise sensors 220 having any number of sampling or measurement storage techniques and that processor 240 may be configured by machine readable instructions 252 to poll sensors 220 having such various sampling or measurement storage techniques.

Machine readable instructions 252 may specify a polling frequency for each sensor 220 or may specify a common polling internal all or a sub-set of sensors 220. As used herein, a polling frequency may specify how often processor 240 polls one or more sensors 220.

In one embodiment, machine readable instructions 252 may define conditions under which the polling frequency associated with one or more sensors 220 may change. For example, machine readable instructions 252 may instruct processor 240 to increase the polling frequency and/or sensor sampling frequency of a temperature sensor 222 in the event that the animal temperature exceeds a threshold value. Instructions 252 may instruct processor 240 to decrease the polling frequency and/or sensor sampling frequency of the temperature sensor 222 if the animal temperate is maintained below the threshold value. Processor 240 may adapt the polling frequency and/or sensor sampling frequency to changing animal health conditions so that potential health risks and/or other changes in animal health state may be recognized as soon as possible while minimizing extraneous sensor measurements and message transmissions. The frequency of sensor 220 polling transmission may be configured to change according to animal location. For example, the processor 240 may increase a polling and/or transmission frequency when the animal is in the vicinity of a calving pen and/or hospital pen, and may decrease the polling and/or transmission frequency when the animal is moved out of the pen. Similarly, the sensor polling and/or transmission frequencies may be configured to vary depending an animal schedule. For example, bolus 210 may be configured to transmit measurements during animal milking time (e.g., three (3) times per day).

In one embodiment, processor 240 may transmit sensor measurements obtained by polling sensors 220 via data transmitter 232. In one mode of operation, processor 240 may form a message comprising the measurements as sensor 220 readings are obtained (after polling the one or more sensors 220). Such a message may be referred to as an animal characteristics message and may be comprised of the sensor readings obtained by polling one or more sensors 220. This operational mode may be referred to as “instantaneous” mode since sensor readings are transmitted as they are polled by processor 240. In another mode of operation, processor 240 may not immediately transmit the sensor readings polled from sensors 220, but instead store them on memory unit 250. In this mode, machine readable instructions 252 may specify a transmission internal, wherein processor 240 may transmit an animal characteristics message comprising some or all of the measurements stored in memory unit 250 at each transmission interval. This operational mode may be referred to as “burst” mode since sensor 220 readings are transmitted as periodic bursts rather than when sensor polling takes place. Operation in “burst” mode may reduce the power consumed by bolus 210 by reducing the number of transmissions sent from data transmitter 232.

In one embodiment, messages transmitted via data transmitter 232 of communications unit 230 may comprise a media access control (MAC) value. A MAC may be a six (6) or three (3) byte value used to uniquely identify messages originating from a particular bolus 210. A MAC address may also be used by data receiver 232 and/or processor 240 to identify messages intended for bolus 210. As such, receiver 232 and/or processor 240 may disregard any incoming messages having a MAC address other than its own, obviating the need to time-slice or otherwise manage wireless traffic between bolus 210 and a base station or other wireless device. MAC addressing to route and control network messages is generally known within the networking arts.

In one embodiment, a programmable unique animal identifier (UAID) may be stored on memory unit 250. In this embodiment, the UAID may be used to associate a bolus 210 with a particular animal. The UAID value may be transmitted with some or all of the messages originating from a particular bolus 210, allowing the receiver of such messages to associate the received data with a particular animal.

In one embodiment, the bolus memory unit 250 may comprise read-only storage 254. Read-only storage 254 may be a Programmable Read-Only Memory (PROM), Erasable Programmable Read-Only Memory (EPROM), Electrically Erasable Programmable Read-Only Memory (EEPROM), or the like. In this embodiment, a unique bolus identifier value (UBID) may be stored within the read-only storage 254. The UBID value may be transmitted with some of all of the messages transmitted from the bolus 210. In this embodiment, the UBID may provide a tamper-proof identifier to uniquely identify a particular bolus 210.

In one embodiment, communications unit 230 may detect whether bolus 210 is within range of a receiver, such as a base station (not shown) or transceiver (not shown). Processor 240 may cause communications unit 230 to transmit a discovery message at a set interval. This simple message may be referred to as a “ping” and may include one or more of the unique identifiers associated with a particular bolus 10 (e.g., a MAC, UAID, and/or UBID). A base station or transceiver receiving the ping message may be configured to send a short reply message indicating that the ping message was received. In this way, processor 240 may know that it is within wireless range of a base station or transceiver. Upon receipt of a reply message, bolus 210 may be configured to be in “on-line” mode. If bolus 210 does not receive a reply message within a threshold period of time, it may transmit additional ping messages. If a threshold number of retry ping messages have been sent without a reply, bolus 210 may be configured to be in “off-line” mode. Machine readable instructions 252 may include instructions to be executed by processor 240 corresponding to “on-line” and/or “off-line” mode.

Alternatively, communication module 230 may be configured to listen for, rather than transmit, the ping discovery messages discussed above. In this embodiment, a base station (e.g., base station 40 of FIG. 1), may be configured to transmit periodic ping messages. Upon receiving one of these messages through communication module 230, processor 240 may cause bolus 210 operate in “on-line” mode and, if no ping messages are received for a threshold time period, processor 240 may cause bolus 210 to operate in “off-line mode.” This may allow bolus 210 to conserve power and may reduce RF interference potentially caused by transmitting periodic ping messages.

In “on-line” mode, bolus 210 may transmit animal characteristics messages at the “online” transmission frequency specified by machine readable instructions 252. As discussed above, such messages may be transmitted as processor 240 polls sensors 220, or may be transmitted at a periodic transmission interval. The receiver of such messages may be configured to respond with a confirmation message. The confirmation message may be used in the place of a separate ping message in order to decrease the message traffic between bolus 210 and the receiver.

In “off-line” mode, bolus 210 may decrease transmission frequency of messages according to machine readable instructions 252. Additionally, while in “off-line” mode, machine readable instructions 252 may direct processor 240 to deactivate certain sensors 220 in order to conserve power. In “off-line” mode, bolus 210 may continue sending “ping” messages in order to discover when bolus 210 comes back into range of a base station or transceiver unit. In this sense, data transmitter 232 of bolus 210 may be considered to be an active transmitter since bolus 210 may actively transmit animal characteristic messages and may actively detect when a base station or transceiver is in wireless communications range. Bolus 210 may actively transmit animal characteristics and/or detect wireless communications without requiring interrogation by an external source.

In one embodiment, bolus 210 may receive new and/or modified machine readable instructions 252 via data receiver 234 of wireless communications unit 230. Such received instructions may comprise changes to the operation of sensors 220 and/or processor 240 including: sensor sampling frequency; sensor reading duration; sensor activation status; sensor calibration data; processor polling frequency; processor operational mode (i.e., “instantaneous” or “burst); and the like.

The embodiment of FIG. 2 may comprise power source 260 coupled to each of sensors 220, communications unit 230 including data transmitter 232 and data receiver 234, processor 240, memory unit 250, and any other power-consuming component of bolus 210. Power source 260 may comprise a battery energy storage device 262, such as a lithium ion battery, lead acid battery, nickel cadmium battery, or the like. In another embodiment, power source 260 may comprise a generator 264. In one embodiment, generator 264 may be a piezoelectric generator or mass/alternator generator to generate power from the movement or vibration of bolus 210 within a host animal. In another embodiment, generator 264 may be a heat-activated generator to generate electrical energy from the body heat of a host animal. In some embodiments, generator 264 may be disposed outside of the bolus enclosure 205. Power source 260 may comprise both battery power storage 262 and generator 264; in this embodiment, power generated by power generator 264 may be stored in battery power storage 262.

Referring again to FIG. 1, one or more bolus 10, 20 may be in wireless communication with a base station 40. As such, bolus 10, 20 may periodically transmit animal characteristics messages to the base station 40. Base station 40 may be communicatively coupled to and/or may comprise a general and/or special purpose computing device 42. This computing device 42 may be configured to create one or more animal profiles associated with one or more animals 12, 22. The device may be further configured to compare any animal characteristics messages to one or more stored animal profiles. As a result of this comparison, the computing device 42 may modify the configuration of the bolus 10, 20 (e.g., modify the bolus 10, 20 polling frequency, sample time, transmission time or the like), may detect a health condition in an animal 12, 22 (e.g., detect an estrus and/or off-feed condition in an animal 12, 22), establish a profile and/or baseline characteristics for an animal 12, 22 or group of animals, or the like.

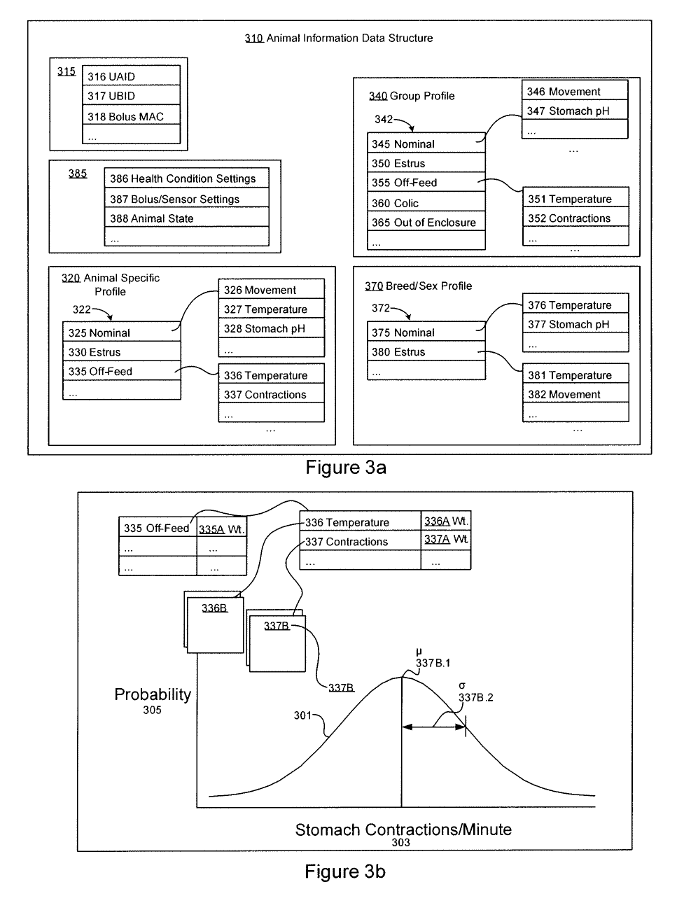

Turning now to FIG. 3a, one embodiment of an animal information data structure 310 is depicted. In the FIG. 3a embodiment, an animal information data structure 310 may comprise animal-identifying information 315. Such information may comprise a unique bolus identifier (UBID) of a bolus and/or sensor associated with animal, a unique animal identifier (UAID), and/or a media access control (MAC) value associated with the bolus and/or sensor. As discussed above, these values may be stored in and/or associated with a bolus and/or sensor associated with the animal. Animal profile 310 may comprise any other animal identifying information known in the art (e.g., an animal name, group breed and/or sex indicator, or the like).

As discussed above, animal characteristics messages transmitted from a bolus and/or sensor (not shown) may comprise one or more of the identifiers discussed above (e.g., UBID, UAID, MAC, or the like). As such, a recipient of the animal characteristics message may be able to associate the message with an animal information data structure 310 using the animal-identifying information 315. For example, in one embodiment, one or more animal information data structures 310 comprising identifying information 315 may be stored and/or managed in a relational database implementing a query language, such as Structured Query Language (SQL). In this embodiment, identifying information 315 may be used as a key (i.e., SQL primary key) into a particular record (i.e., animal information record 310) in the relational database. This may allow a particular animal information data structure 310 to be obtained from one or more animal identifier(s) 315. In alternative embodiments, identifying information 315 may be used in other ways to obtain animal information data structure 310. For instance, in an embodiment using a lightweight directory protocol (LDAP) data storage system, identifying information 315 may comprise directory naming information and/or LDAP search parameters. One skilled in the art would recognize that the animal information data structure 310 of this disclosure should be stored and/or managed using any data storage and/or management technique known in the art. As such, this disclosure should not be read as limited to any particular data storage and/or management technique.

Animal information data structure 310 may comprise an animal-specific profile 320. Animal-specific profile 320 may comprise profile information relating to a particular animal. Animal-specific profile 320 may comprise one or more animal health-condition patterns 322 including, but not limited to, a nominal (i.e., normal and/or healthy) heath-condition pattern 325, an estrus health-condition pattern 330, an off-feed health-condition pattern 335, and the like. Each animal health-condition pattern 322 may comprise one or more animal characteristic models corresponding to a health condition (e.g., movement 326, temperature 327, stomach pH 328, etc.)

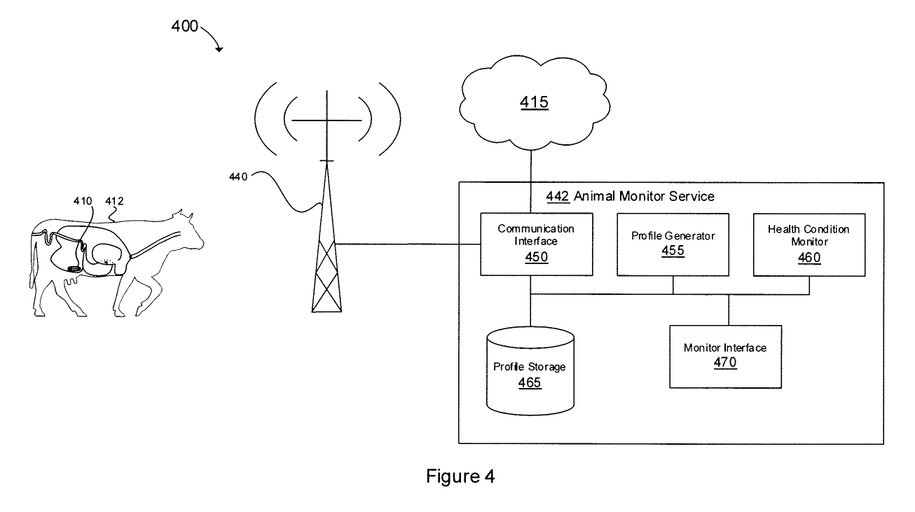

Each animal health-condition pattern 325, 330, 335 may comprise one or more animal characteristic models corresponding to the health condition. The animal characteristic models may relate to any measurable animal characteristic (i.e., any characteristic the bolus and/or sensor are capable of measuring). For instance, FIG. 3a depicts nominal animal health-condition pattern 325 as comprising, inter alia, an animal movement model 326, animal temperature model 327, and a stomach pH model 328. Models 326, 327, 328 may be statistical models created by observing the animal when known to be in a nominal health condition (e.g., when healthy). A process for generating models, such as 326, 327, and 328, is discussed in detail below in conjunction with FIG. 4. The animal characteristic models of animal information data structure 310 may allow the system to detect a particular animal health condition. For example, the nominal health condition 325 may be detected if measured animal characteristics correspond to animal characteristic models 326, 327, 328 within a health condition detection threshold.

Animal information data structure 310 may comprise a group profile 340. Group profile 340 may comprise animal profile information relating to a particular group of animals (e.g., animals in a particular feedlot or dairy). Animals in a group may exhibit one or more common characteristics. For example, the animals in the group may have a common schedule (e.g., fed at a particular time and/or milked or otherwise processed on a particular schedule). As such, the animals in the group may exhibit increased movement activity during these common feeding and/or processing occurrences. Other animal characteristics may vary depending upon group behavior. For instance, an animal may experience short-term, measured, temperature drops during watering. This may occur since the bolus temperature sensor may be disposed in the animal's stomach. As the animal drinks, its measured body temperature may be reduced for a short time.

A group profile 340 may be useful where an animal manager has only created animal-specific profiles for only a few of the animals in a particular group (e.g., an animal sub-set). This may occur since generating an animal profile may require monitoring the animal over time, and animals may come and go from a particular group before a profile may be developed. In addition, the animal manager may find that a group profile detects animal health conditions with sufficient accuracy, and the development of animal specific profiles may be unnecessary. Moreover, since the group profile data may comprise data from a number of different animals over a relatively long time period (compared to a particular animal in the group), a group profile may yield better results and/or represent a simply low-cost alternative to generating animal-specific profiles 320 for all the animals in a particular group.

Although group profile 340 is depicted as part of animal information data structure 310, one skilled in the art would recognize that since group profile 340 may contain data common to multiple animal information data structures 310, its inclusion in an animal information data structure 310 may be implemented as a link, reference, or any other indirect data relation technique known in the art to avoid unnecessary replication of group profile 340 data.

As with the animal specific profile 320 discussed above, group profile 340 may comprise one or more animal health-condition patterns 342. Heath-condition patterns 342 may correspond to various animal health conditions including, but not limited to: a nominal 345 pattern; an estrus 350 pattern; an off-feed 355 pattern; a colic 360 pattern; an out-of-enclosure 365 pattern (i.e., detect that the animal as left the group enclosure/pen); and the like. As discussed above, each health-condition pattern 342 may comprise one or more animal characteristic statistical or other models relating one or more animal characteristics to the health condition. For example, the out-of-enclosure pattern of 365 may comprise an animal position model (e.g., geofence) indicating that if the animal is outside of a particular area, the animal is outside of the enclosure defined for a particular animal group. Group profile 340 may comprise nominal health-condition pattern 345 comprised of a movement 346 and stomach pH 347 animal characteristic models and off-feed pattern 355 may comprise temperature 351 and stomach contractions 352 animal characteristic models.

Animal information data structure 310 may comprise a breed/sex profile 370. Animal breed/sex profile 370 may comprise health-condition patterns 372 common to a particular breed and/or sex of animal (e.g., Holstein dairy cows). Animals of a particular breed and/or sex may experience similar physiological markers when in a particular health condition. For example, Holstein dairy cows may have common baseline characteristics (e.g., movement, stomach pH, temperature, etc.). As such, a breed/sex profile may include a nominal health-condition pattern 375. Similarly, animals of a particular breed/sex may exhibit common estrus-condition characteristics. For example, Holstein dairy cows may experience, inter alia, a one (1) degree Fahrenheit increase in body temperature and increased movement activity when in an estrus health condition. Accordingly, breed/sex profile 370 may comprise an estrus health-condition pattern 380 comprising an animal temperature characteristic model 381 and animal movement characteristic model 382 reflecting these changes. Animal breed/sex profile 370 may comprise a nominal health-condition pattern 375 comprised of temperature 376 and stomach pH 377 animal characteristic models and estrus health-condition pattern 380 comprised of temperature 381 and movement 382 animal characteristic models.

A breed/sex profile 370 may be useful where an animal manager is responsible for a large number of animals and generating an animal-specific and/or group profile is not efficient and/or a breed/sex profile is sufficiently effective at detecting animal health conditions. Moreover, a breed/sex profile 370 may be well developed since it may comprise animal characteristic data from a large number of animals observed over relatively long time period. Furthermore, a breed/sex profile 370 may be available from a third party (e.g., the animal monitoring system provider) and may be ready for use immediately, whereas an animal-specific profile 320 and/or group profile 340 may require an initialization and setup period for generating animal health condition models.

An animal information data structure 310 according to the teachings of this disclosure may comprise all three profiles 320, 340, 370, and/or a subset of one (1) or two (2) of the profiles 320, 340, and 370, or additional profiles (e.g., an animal regional profile (a profile corresponding to animals in a particular region), or the like). For example, where an animal is newly integrated into a herd and/or group, an animal information data structure 310 associated with the animal may only comprise a group profile 340 and breed/sex profile 370, until the animal has been observed and animal-specific data gathered. In other cases, where the animal is not expected to be a member of the group and/or herd for a significant amount of time, an animal-specific profile 320 for the animal may never be generated.

Although, FIG. 3a depicts several animal health conditions (e.g., nominal, estrus, off-feed, colic, out of enclosure, etc.), one skilled in the art would recognize animal information data structure 310 may comprise animal health condition patterns corresponding to any animal health condition including, but not limited to: animal stress; lameness; richness of feed; conception; calving or the like.

A particular animal, group of animals, and/or breed may exhibit increased movement activity when stressed. Stress may be caused by various factors, including being integrating an animal into a group after calving, moving the animals, environmental conditions, harassment by other animals or humans, or the like. When stressed, the animal(s) milk production and/or weight gain may decrease. If the stress condition persists, an animal manager may wish to move the animal into another enclosure or otherwise address the source of the stress. Accordingly, a stressed animal health-condition pattern may be developed to detect a stressed animal based on its movement characteristics.

Data structure 310 may further comprise an animal health-condition pattern corresponding to animal lameness. Reduced animal movement activity may indicate that the animal has become lame. An animal manager may wish to detect such a condition to save the animal and/or prevent it from becoming permanently lame. For example, if the lameness is caused by a wound, the condition may worsen over time, may become infected, and/or may pose a serious risk to the animal. Accordingly, a lame animal health-condition pattern may be developed to detect a lame animal based on its movement and/or motion activity characteristics.

A richness of feed condition may be detected by modeling an average temperature of a ruminant animal's stomach. For some animals and animal breeds, the “richness” of feed (e.g., the corn content of the feed) may affect the animal's internal stomach temperature. A feed that is too rich may result in a high internal temperature and may “burn out” the animal, which may reduce animal weight gain and/or animal milk production. Accordingly, a “rich feed” animal health-condition pattern may be developed to detect feed richness problems to thereby allow an animal manager to modify the feed formulation.

As discussed above, an animal health-condition pattern may be used to detect an animal estrus health-condition. Additional patterns may detect other reproductive changes in the animal. For example, a “conception” pattern may detect an end to an animal's estrus cycle. In some animals, upon conception, the animal's internal temperature may drop and its movement and/or motion activity characteristics may change. As such, a conception animal health-condition pattern may be developed to detect this condition. Similarly, birthing (i.e., calving) may cause changes to movement and/or motion activity, temperature, and stomach contraction activity. Accordingly, a birthing health-condition pattern may be developed to detect conception and/or birthing activity in the animal.

Turning now to FIG. 3b, one embodiment of a health-condition pattern 335 and corresponding animal characteristic model 337 is depicted. The heath-condition pattern 335 depicted in FIG. 3b may correspond to an off-feed health condition. Off-feed health-condition pattern 335 may comprise animal characteristics models 336 and 337 corresponding to animal temperature 336 and stomach contractions 337 respectively. As discussed above, animal drinking behavior may be detected by monitoring a periodic, temporary drop in internal temperature due to drinking activity (e.g., as the animal drinks, water may enter the animal's stomach, causing a drop in measured animal temperature). In addition, while on-feed, a ruminant animal may experience approximately three stomach contractions per minute. Changes to these animal characteristic models may be captured in an off-feed animal-condition pattern 335. For example, the temperature 336 model of the off-feed health condition pattern may omit some or all of the temporary temperature dips expected due to drinking. As such, rather than modeling an expected value of animal temperature, temperature model 336 may model the temporary temperature dips due to drinking activity. Similarly, off-feed stomach contraction model 337 may model a reduction in the expected rate of stomach contractions and/or may comprise an inverse probability model of nominal behavior (e.g., three contractions per minute).

The temperature 336 and stomach contractions 337 animal characteristic models may comprise one or more statistical models (336B and 337B). For example, temperature model 336 may comprise one or more statistical models 337B, and stomach contractions model 336A may comprise one or more statistical models 337B.

The statistical model(s) associated with temperature 336 and stomach contractions 337 may be circadian, in that a 24-hour cycle is monitored. A circadian profile may be used since animal behavior, and corresponding animal characteristics, may differ significantly during the day. For example, an animal may sleep during evening hours and, as such, may not drink nor exhibit periodic temporary temperature drops due to the drinking. As such, different models may be used to monitor animal behavior depending on the time of day (e.g., a circadian model). Similarly, stomach contractions may differ depending upon the time of day. For example, if the animals are fed at a particular time, contraction activity may increase during that time. Similarly, while the animal is sleeping, contraction activity may change.

FIG. 3b depicts one embodiment of a statistical model 337B to model off-feed animal stomach contraction behavior. Stomach contraction model 337B may comprise a plurality of normal (i.e., Gaussian) probability distributions. The distributions may correspond to different times of day to create a circadian profile of stomach contraction behavior.

Where a normal distribution is used, each statistical model of 337B may comprise a mean μ 337B.1 and standard deviation σ 337B.2. Animal stomach contraction activity may be mapped onto the stomach contractions-per-minute axis 303. This axis 303 may then yield a probability value depicted on probability axis 305. The resulting probability may be the probability that the observed stomach contraction characteristic data was produced by an animal having and/or in an off-feed health condition. Where a normal distribution 301 is used, this probability may be calculated using a normal probability density function according to Equation 1.3:

$$p = \frac{1}{σ \sqrt{2 π}} exp ( - \frac{{( x - μ )}^{2}}{2 {σ}^{2}} ) \qquad Eq . 1.3$$

In Equation 1.3, x may represent the observed animal movement and/or motion activity characteristics, μ may represent the mean of an off-feed stomach contractions model 337B.1, and a may be the standard deviation of the estrus movement model 337B.2. In an alternative embodiment, model 337B may model nominal behavior (e.g., three contractions per minute); as such, the resulting probability may be inverted to determine a probability that the animal is off-feed.

One skilled in the art would recognize that other statistical models could be used under the teachings of this disclosure including, but not limited, to: a Rayleigh distribution; chi-square distribution; Cauchy distribution; or the like. In addition, other pattern matching techniques could be used including, but not limited to: abstract tree matching; Baysian pattern matching; regular expression matching; or the like. As such, this disclosure should not be read as limited to any particular distribution type and/or pattern matching technique.

As depicted in FIG. 3b, a health condition pattern 335 may comprise a proportional weight 335A. Proportional weight 335A may define a proportional weight for the health condition pattern 335 relative to other health condition patterns in other profiles (e.g., group 340 and/or breed/sex 370 profiles). For example, off-feed health condition pattern 335 may be part of an animal-specific profile 320. Group profile 340 may include an off-feed health condition pattern 355 as may breed/sex profile 370 (not shown). When detecting an animal health condition, the animal health condition profiles from each of the animal-specific profile 320, group profile 340, and breed/sex profile 370 may be used. Each health condition pattern may yield a probability for the same health condition. For instance, for the nominal health condition, the animal-specific profile 320 may yield a nominal health-condition 325 probability P_{AS.Nom}, the group profile 340 may yield a nominal health-condition 345 probability P_{G.Nom}, and the breed/sex profile may yield a nominal health-condition 375 probability P_{BS.Nom}. On overall nominal health-condition probability, P_{Nom}, may be calculated by averaging the individual profile probabilities per Equation 1.4:

$${P}_{Nom} = \frac{1}{3} ( {P}_{AS , Nom} + {P}_{G , Nom} + {P}_{BS , Nom} ) \qquad Eq . 1.4$$

Alternatively, the probabilities of each profile may be proportionately weighed. Such proportional weighing may be used where a particular animal profile (e.g., animal-specific profile) is thought to a better predictor of the health condition. In this case, the overall probability P_{Nom} may be calculated per Equation 1.5:

$${P}_{Nom} = \frac{1}{3} ( {W}_{AS , Nom} {P}_{AS , Nom} + {W}_{G , Nom} {P}_{G , Nom} + {WS}_{BS , Nom} {P}_{BS , Nom} ) \qquad Eq . 1.5$$

In Equation 1.5, W_{AS.Nom} may be the proportional weight given to the nominal health condition pattern 325, W_{G.Nom} may be the proportional weight given to the nominal health condition pattern 345, and W_{BS.Nom} may be the proportional weight given to the breed/sex nominal health condition pattern 375.

Referring again to FIG. 3b, each animal characteristic model 336 and 337 of a particular health condition pattern 335 may comprise a proportional weight for the characteristic (336A, 337A). These weights may be used where a particular animal characteristic is deemed to be a better indicator and/or predictor of a particular health condition. For example, in FIG. 3b, stomach contractions 337 may be found to be a better predictor of an off-feed condition 335 than temperature 336. As such, the weight 336A may be proportionally greater than weight 337A. As described above, where the weights are equal (or not used), the probability of a particular health condition may be calculated per Equation 1.6:

$${P}_{AS , Off \text{-} Feed} = \frac{1}{2} ( {P}_{Temp , Off \text{-} fe ed} + {P}_{Contract , Off \text{-} feed} ) \qquad Eq . 1.6$$

In Equation 1.6, the probability of the animal-specific profile 320 off-feed health-condition pattern 335, P_{AS.Off-feed}, may be calculated as an average of the temperature 336 and contractions 337 probabilities. Where weights are used, the temperature probability P_{Temp.Off-feed} and contractions probability P_{Contract.Off-feed} may be scaled by a proportional weighing factor substantially as shown above in Equation 1.5.

Referring again to FIG. 3a, a particular animal characteristic model (e.g., stomach contractions model 337) may comprise multiple models to comprise a circadian model (e.g., models covering time period such, as a day). An animal characteristic model may comprise additional, animal characteristic models corresponding to other factors, such as the time of year, season, or the like. For example, the animal movement and/or motion activity, such as nominal animal movement model 346 in group profile 340, may be circadian and, as such, may comprise a movement profile for each hour, or other time period, in a given day to take into account the animal's routine (e.g., sleep, feeding, and/or processing schedule). In addition, movement model 346 may vary depending upon ambient conditions. For example, when the ambient temperature is low or high, the animal's movement profile may differ. Similarly, weather conditions may affect animal movement and/or motion activity. As such, movement model 346 may comprise various statistical movement models developed for each of these differing conditions.

Animal information data structure 310 may further comprise monitor settings 385. Monitor settings 385 may comprise health condition settings 386. Health condition settings 386 may comprise a probability detection threshold and time threshold associated with each animal health condition (e.g., may define a minimum probability and a minimum time the probability must be maintained in order to detect a particular health condition). In addition, health condition settings 386 may comprise directives to define actions to be taken by an animal-monitoring system upon detecting a particular health condition in an animal (e.g., immediately notify/alert an animal manager, etc.). For instance, health condition settings 386 may comprise a directive to alert the animal manager via a text message, voice message, email, audible alert, or the like if an animal goes off-feed, enters or exits an estrus condition, or the like. For less critical health conditions (e.g., nominal), health condition settings 386 may not include a notification and/or alerting directive. As with the group 340 and breed/sex 370 profiles, health condition settings 386 may be shared across multiple animal information data structure 310. As such, health condition settings 386 may be indirectly referenced and/or linked to animal information data structure 310.

Monitor settings 385 may comprise sensor settings 387. Sensor settings 387 may include information relating to the sensor and/or bolus associated with the animal data structure 310. Such information may include the operational mode of the bolus (e.g., burst and/or instantaneous mode), a polling frequency of the bolus and/or bolus sensors, a sampling frequency of the bolus and/or sensors, a sensor read duration of the bolus sensors, sensor calibration data, and the like.

Monitor settings 385 may comprise animal state data 388. Animal state data 388 may include information relating to the current health condition(s) of the animal (e.g., nominal, estrus, off-feed, out-of-enclosure, or the like). In addition, animal state data 388 may include past sensor readings and/or past health condition probabilities and probability durations (i.e., how long a particular probability has been maintained at a particular level). Such information may be used by the animal health condition, monitoring system in detecting one or more health conditions. For example, detecting some animal health conditions may require that certain animal characteristics be maintained for a threshold time period before the associated health condition is detected. Animal state data 388 may further be used to detect animal health condition changes. For example, animal state data 388 may indicate that an animal is in an estrus state. If the monitoring system subsequently detects that the animal is in a nominal (i.e., non-estrus condition), the system may detect the change, and the health condition settings 386 discussed above may direct that an animal manager be notified and/or alerted to the change.

Although animal information data structure 310 is depicted as a block data structure, such as those used in a relational data base, one skilled in the art would recognize that there are other ways of storing, representing, and managing such data known in the art, any of which could be used under the teachings of this disclosure. As such, this disclosure should not be read as limited to any particular data representation, storage, and/or management technique.

Turning now to FIG. 4, a block diagram of a system 400 for monitoring a health condition of an animal is depicted. System 400 may comprise a base station 440 in wireless communication with a bolus 410 disposed within an animal 412. Base station 440 may comprise an animal monitor service 442 and/or may be communicatively coupled to animal monitor service 442. Animal monitor service 442 may comprise a general or special purpose computing device. As such, animal monitor service 442 may comprise an application running in conjunction with a computer operating system (OS), such as Microsoft® Windows™, Linux®, Apple OS.X®, or the like. The general or special purpose computing device of animal monitor service 442 may comprise an input/output (I/O) system comprising a display, audio output, pointer input, keyboard input, or the like.

Messages wirelessly received from bolus 410 by base station 440 may be sent to communication interface 450 of animal monitor service 442, and messages sent to bolus 410 through communication interface 450 of animal monitor service 442 may be wirelessly transmitted to bolus 410 by base station 440.

Communication interface 450 may be in communication with base station 440 and a network 415. Network 415 may comprise any communications network known in the art including, but not limited to: a local area network (LAN); a wide area network (WAN); a Wireless Fidelity® and/or Wi-Fi network; a cellular telephone network; a PSTN network; an RF network; a radio network; the Internet; or the like. Accordingly, it should be understood that this disclosure, and specifically network 415, is not limited to any particular communication method and/or protocol and that the teachings of this disclosure may be implemented using any communications method and/or protocol known in the arts. In addition, network 415 may comprise a combination of various network communication systems and/or protocols. As such, network 415 should not be read as limited to any single network communication infrastructure and/or protocol.

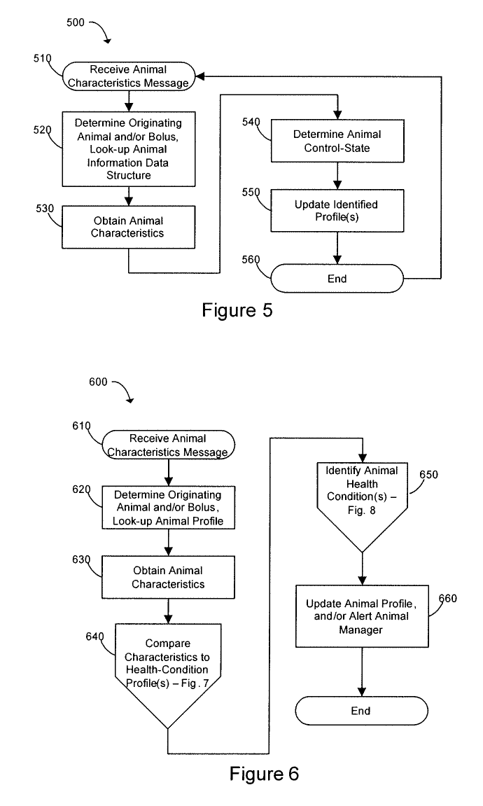

Animal monitor service 442 may comprise a profile generator module 455. Profile generator module 455 may establish, update, and otherwise manage animal information data structures per FIGS. 3a and 3b and, in particular, establish animal profiles and health-condition patterns. One embodiment of a process for generating and/or updating an animal-specific, group, and/or breed sex profile and related health-condition patterns is described below in conjunction with FIG. 5.

Animal monitor service 442 may comprise health-condition monitor module 460. Health-condition monitor module 460 may monitor animal characteristics received from bolus 410 and compare the received characteristics to one or more animal profiles and related health condition patterns stored, created, and/or managed by profile generator 455 and stored in profile storage 465 (e.g., an animal information data structure 310 of FIG. 3a). Health-condition monitor module 460 may be communicatively coupled to communication interface 450. User preferences associated with one or more animal health conditions may specify that, upon detecting the health condition in an animal, an animal manager should be notified. Upon detecting such a health condition, health-condition monitor 460 may send an alert to an animal manager via communications interface 450. In addition, detecting animal characteristics within a certain threshold of an animal health condition may require the configuration and/or settings of bolus 410 to be changed (e.g., increase and/or decrease the polling frequency, operational mode, sensor configuration, or the like). As such, health-condition monitor module 460 may use communication interface 450 to send bolus configuration messages to bolus 410 via base station 440.

Profile storage 465 may comprise data storage means for storing one or more animal information data structures and associated animal profiles, health condition patterns, and animal characteristics models. The data stored therein may correspond to the animal information data structure described above in conjunction with FIGS. 3a and 3b. Profile storage 465 may comprise any data storage and retrieval means known in the art including, but not limited to: a fixed disk (e.g., hard drive); flash memory; optical memory; a relational database; a flat file system; an XML database; an X.509 directory; a lightweight directory access protocol (LDAP) directory service; or the like. One skilled in the art would recognize any data storage and/or retrieval mechanism known in the art could be under the teachings of this disclosure. As such, this disclosure should not be read as limited to any particular data storage and/or retrieval system.

Animal monitor service 442 may comprise monitor interface 470. Monitor interface 470 may allow an animal manager, or other service 442 operator, to configure and otherwise manage animal monitor service 442. As such, monitor interface 470 may comprise an application interface. Monitor interface 470 may be network and/or web-accessible. As such, monitor interface 470 may be accessible via network 415 and/or utilize I/O capabilities of the special or general purpose computing system of animal monitor 442.

Turning now to FIG. 5, one embodiment of a method 500 for establishing an animal health condition pattern and/or one or more associated animal characteristic models is depicted. As discussed above, a base station according to the teachings of this disclosure may comprise and/or be in communication with a general and/or special purpose computing device, such as a personal computer running OS. Accordingly, the steps of the flow diagram 500 may be executed by a computing device and/or comprise one or more computer readable instructions to be executed by the computing system. Such instructions may be stored on a computer readable medium, such as a CD, fixed disk, memory unit, or the like. Alternatively, the steps of flow diagram 500 (or the other flow diagrams contained herein) may be performed in hardware, such as in a state machine, field programmable gate array (FPGA), application specific integrated circuit (ASIC), an electrically erasable read only memory (EEPROM), or the like.

At step 510, an animal characteristics message from a bolus and/or sensor associated with an animal may be received. The bolus may be disposed within the stomach (e.g., rumen and/or reticulum of a ruminant animal). The animal characteristics message may be received wirelessly by one or more antennae communicatively coupled to a base station. The base station may be configured to forward such messages to process 500 running in conjunction with the general and/or specific purpose computing device communicatively coupled to the base station.

At step 520, an identifier may be obtained from the animal characteristics message. In one embodiment, this step may be performed by reading a unique identifier from the message (e.g., a bolus identifier (UBID), unique animal identifier (UAID), media access control (MAC) value, or the like). The identifier may be used to look-up the corresponding animal information data structure in a data storage system (e.g., relational database, flat-file, profile storage 465 of FIG. 4, etc.). The data obtained at step 520 may comprise an animal information data structure described above in conjunction with FIGS. 3a and 3b.

At step 530, one or more animal characteristics may be obtained from the animal characteristics message. As discussed above, such animal characteristics may comprise measurements corresponding to the internal physiological state of the animal (e.g., temperature, stomach contractions, stomach pH, etc.) and/or measurements corresponding to other animal characteristics (e.g., animal movement and/or motion activity, animal position, etc.). The animal characteristics obtained at step 530 may comprise the instantaneous readings of one or more bolus sensors if the source bolus is operating in “instantaneous” mode and/or may comprise a series of readings if the source bolus is operating in “burst” mode.

At step 540, a control state of the animal may be identified. The control state of step 540 may correspond to an animal health condition discussed above in conjunction with FIG. 3a. For example, the control state of step 540 may be a nominal, off-feed, and/or estrus health condition, or the like as discussed above in conjunction with FIG. 3a. The control state of the animal may be specified by an animal manager upon observing the animal. As such, the animal characteristics obtained at 530 may be used to develop one or more animal characteristics indicative of the identified control state (e.g., estrus, off-feed, nominal, etc.).

At step 550, the animal characteristics obtained at step 530 may be used to update one or more animal characteristic models associated with the health condition, control state of step 550. As discussed above, a health condition pattern (e.g., nominal, off-feed, estrus, etc.) may comprise one or more animal characteristic models (e.g., animal movement and/or motion activity, stomach contractions, temperature, or the like). The models may be used to later detect the animal health condition. Accordingly, the animal characteristics of step 530 may be used to update the models of the animal control state identified at 540. For example, if the animal control state of step 540 is an estrus state, models corresponding to animal movement and/or motion activity, temperature, and the like may be updated. Updating may comprise updating a statistical model associated with the particular animal characteristic. As discussed above, animal characteristics may be modeled using a probability distribution function, such as a normal (i.e., Gaussian) distribution. Where a normal distribution is used, the animal characteristics of step 540 may be used to calculate the distribution mean μ and standard deviation σ.

Equation 1.7 depicts one way of calculating a normal distribution mean:

$$μ = \frac{1}{N} {\sum }_{i = 1}^{n} {x}_{i} \qquad Eq . 1.7$$

Equation 1.7 may calculate a mean value μ for the animal characteristic. In Equation 1.7, xᵢ may represent an animal characteristic obtained at step 530, and N may be the number of samples comprising the model (i.e., the total number of animal characteristic samples obtained to that point). Alternatively, the mean may be calculated incrementally per Equation 1.8:

$${μ}_{i} = \frac{N * {μ}_{i - 1} + {x}_{i}}{( N + 1 )} \qquad Eq . 1.8$$

In Equation 1.8, μᵢ₋₁ may represent the distribution mean before inclusion of the current sample, xᵢ, and μᵢ may represent the distribution mean after inclusion of xᵢ.

The standard deviation of a normal distribution model may be calculated per Equation 1.9:

$$σ = \sqrt{\frac{1}{N} {\sum }_{i = 1}^{n} {( {x}_{i} - μ )}^{2}} \qquad Eq . 1.9$$

In Equation 1.9, xᵢ may be the current animal characteristic value obtained at step 530, N may be the number of samples currently comprising the model, and μ may be the model mean.

As discussed above in conjunction with FIG. 3a, an animal information data structure may comprise and/or be linked to an animal-specific profile, a group profile, and/or a breed/sex profile. In this embodiment, the models corresponding to the specific animal, group, and/or breed/sex may be updated at step 550 in like fashion. Alternatively, where an animal specific profile does not exist, only the animal group profile and/or animal breed/sex profile may be updated at step 550.

At step 560, the flow may terminate and/or wait for additional animal characteristics messages to be received at step 510.

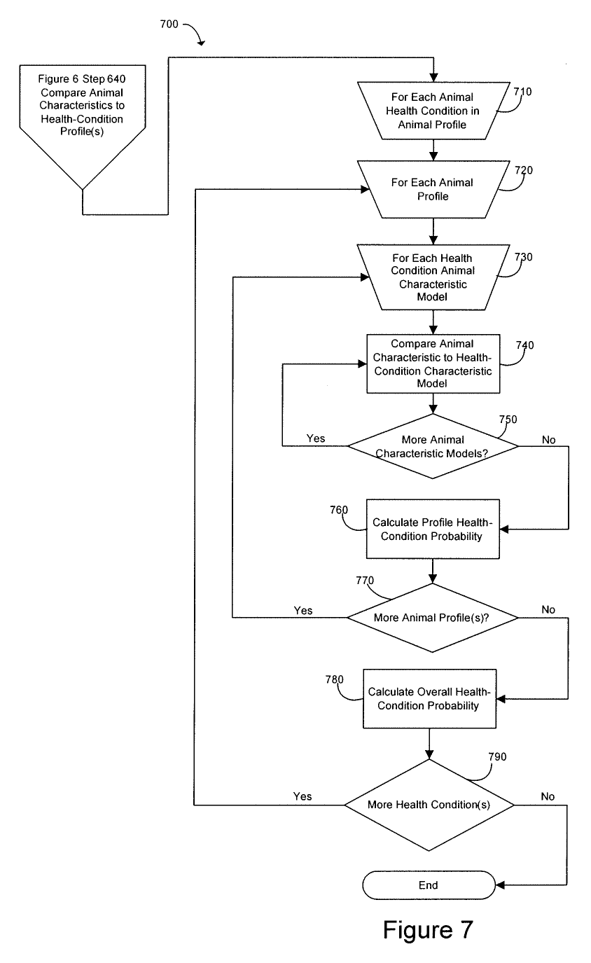

Turning now to FIG. 6, a flow diagram of one embodiment of a method 600 for detecting a health condition in an animal is depicted.

At step 610, an animal characteristics message may be received. As discussed above, the computing device executing the steps of method 600 may be communicatively coupled to a base station according to the teachings of this disclosure. The base station may be in wireless communication with one or more sensors and/or bolus monitoring one or more animals. The base station may be configured to forward messages comprising animal characteristics to method 600.

At step 620, an animal profile associated with the message may be determined. As discussed above, the animal characteristics message received at step 610 may comprise one or more unique identifiers. The one or more identifier(s) may be obtained from the message and used to lookup or otherwise obtain an animal information data structure (i.e., a data structure 310 depicted in FIG. 3a).

At step 630, one or more animal characteristics may be obtained from the message. The animal characteristics of step 630 may include one or more samples of one or more internal physiological and/or other animal characteristics.

At step 640, the animal characteristics obtained at step 630 may be compared against one or more animal health condition patterns in one or more animal profiles (e.g., an animal-specific profile, a group profile, and/or a breed/sex profile). These profiles may be stored in and/or linked to animal information data structure obtained at step 620. As discussed above, an animal health condition may comprise one or more animal characteristic models. Accordingly, the comparing of step 640 may comprise comparing the animal characteristics of step 630 to each of these animal characteristic models to determine a probability that the animal has/is in a particular health condition. The comparing may further comprise combining the comparison results from an animal-specific profile, group profile, and/or breed/sex profile where such are available. The probabilities obtained from each comparison may be combined to form an overall probability that the animal has/is in a particular health condition. This combination may be proportionally weighted based upon the animal characteristics received and the corresponding animal profiles comprising the comparison. One embodiment of a process 700 for making the animal characteristics to health condition comparison of step 640 is described below in conjunction with FIG. 7.

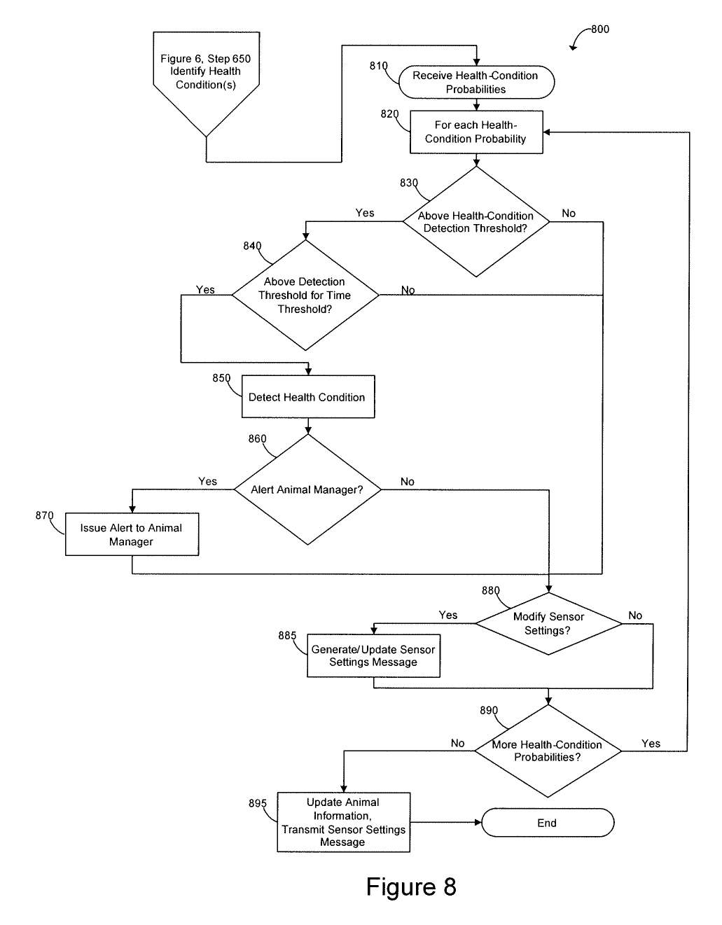

At step 650, the result of the animal characteristics to animal-condition comparisons may be evaluated to detect one or more health conditions in the animal. Detecting an animal health condition may comprise determining whether the probability that an animal is in a particular health condition is above a probability detection threshold associated with the health condition. The detecting may further comprise determining whether the threshold has been maintained for a threshold period of time (e.g., maintained for two (2) hours). If so, method 600 may detect that the animal is in/has the health condition. One embodiment of a method 800 for detecting a health condition is described below in conjunction with FIG. 8.

After identifying one or more animal health conditions, the flow may continue to 660. At step 660, the animal profile obtained at step 620 may be updated to reflect the animal health condition(s) identified at step 650. In addition, an animal manager may be alerted to the detected animal health condition per settings in the animal information data structure (e.g., health condition settings 386 of animal information data structure 310 in FIG. 3a).

Turning now to FIG. 7, a flow diagram of a method 700 for comparing animal characteristics to one or more health conditions defined in an animal profile is depicted. Method 700 may comprise part of a method (e.g., method 600 depicted in FIG. 6) for detecting a health condition in an animal.

At step 710, method 700 may loop through each animal health condition in an animal information data structure. As discussed above, an animal information data structure may comprise one or more animal profiles including one or more animal health condition patterns, such as nominal, estrus, or the like. As such, at step 710, a list, or other data structure, of animal health conditions in an animal profile may be iterated such that steps 720-780 are preformed for each health condition. Such a list may be maintained in an animal information data structure (e.g., element 385, 386 of animal information data structure 310 in FIG. 3a).

At step 720, method 700 may loop through each of the profiles in an animal information data structure. As discussed above, an animal information data structure may comprise one or more animal profiles, such as an animal-specific profile, a group profile, a breed/sex profile, or the like. Each of these profiles may comprise health condition model data (e.g., nominal, estrus, off-feed, or the like). As such, method 700 may iterate over all of the available profiles to calculate an overall probability that the animal has/is in a particular health condition.

At step 730, method 700 may loop through each animal characteristic model comprising a particular heath-condition pattern. As depicted in FIG. 3a, a health condition pattern of an animal profile may comprise one or more animal characteristic models. For example, FIG. 3a shows an off-feed health condition pattern 335 of animal-specific profile 320 comprising animal characteristic models for temperature 336 and contractions 337. Therefore, to calculate the probability the off-feed health condition 330 for the animal specific profile 320, each of the temperature 336 and contractions models 337 must be evaluated. As such, at step 730, method 700 may iterate over each of the animal characteristic models defined in the health condition pattern within the animal profile of the animal information data structure.

At step 740, a received animal characteristic may be compared against an animal characteristic model. As described above, an animal characteristic model may comprise a statistical model, such as a normal (i.e., Gaussian) probability distribution having a mean μ and standard deviation σ. As such, the probability that a particular measured animal characteristic corresponds to an animal characteristic of a particular animal health condition may be calculated per Equation 1.10:

$${P}_{temp} = \frac{1}{{σ}_{temp} \sqrt{2 π}} exp ( - \frac{{( {x}_{temp} - {μ}_{temp} )}^{2}}{2 {σ}_{temp}^{2}} ) \qquad Eq . 1.10$$

In Equation 1.10, Pₜₑₘₚ may be the probability that a particular animal characteristic measurement (e.g., animal temperature), xₜₑₘₚ, corresponds a particular characteristic model (e.g., temperature characteristic) of a particular health condition model (e.g., off-feed). As such, μₜₑₘₚ may represent the off-feed health condition temperature characteristic model mean, and σₜₑₘₚ may represent the off-feed health condition temperature characteristic standard deviation.

At step 750, process 700 may determine whether all of the animal characteristics models have been calculated per Equation 1.10. If not, the flow may continue to 740 where the next animal characteristic model may be evaluated. If all of the animal characteristic models for the current animal profile health condition pattern have been evaluated, the flow may continue to step 760.

The successive iteration over step 740 cause method 700 to calculate a probability associated with each of the received animal characteristics (e.g., a probability that the measured characteristic corresponds to the characteristic model in the health-condition pattern). For example, if an off-feed health condition in an animal-specific profile were to comprise a model for animal temperature and stomach contractions, method 700 may iterate over step 740 two times, once for temperature and once for contractions. As such, two probabilities may be calculated, a probability associated with the temperature characteristic model and a probability associated with the contractions characteristic model.

At step 760, the probabilities for each of the characteristic models may be combined. Step 760 may comprise averaging proportionally weighted probabilities of each of the animal characteristic models calculated at step 740 (e.g., probability of correspondence in temperature characteristic and probability of correspondence in contractions characteristic) as shown in Equation 1.11:

$${AP}_{HC} = \frac{1}{N} {\sum }_{i = 1}^{N} {w}_{i} * {p}_{i} \qquad Eq . 1.11$$

In Equation 1.11, AP_{HC} may represent the probability of a particular heath-condition of a particular profile (e.g., animal-specific profile), N may be the number of animal characteristics comprising the health condition pattern, pᵢ may represent a probability corresponding to a particular animal characteristic model, and wᵢ may represent a proportional weight of the animal characteristic relative to other animal characteristic models in the health condition pattern. For example, referring again to FIG. 3a, Equation 1.12 may represent the probability of an off-feed health condition 330 per the animal-specific profile 320:

$${p}_{AS , Off \text{-} feed} \frac{1}{2} ( {w}_{temp} * {p}_{temp} + {w}_{contract} * {p}_{contract} ) \qquad Eq . 1.12$$

In Equation 1.12, “N” of Equation 1.11 is two (2), P_{AS.Off-feed} may represent the probability that an animal is in an off-feed health condition according to an animal-specific profile, pₜₑₘₚ may represent the probability that the measured temperature animal characteristic(s) correspond to the off-feed temperature characteristic model, and p_{contract} may represent the probability that the measured stomach contraction animal characteristic(s) correspond to the off-feed contraction characteristic model. In Equation 1.12, wₜₑₘₚ and w_{contract} may represent proportional weights of the temperature and contraction characteristics models. These weights may be used where one of the characteristics is considered to be a better indicator of the animal health condition than another. For instance, the weight may be proportional and/or derived from the statistical variance of the characteristic model (e.g., less variance may yield more weight and vice versa).

As discussed above, an animal information data structure may comprise multiple animal profiles (e.g., an animal-specific profile, group profile, breed/sex profile, or the like). Accordingly, at step 770, the probability of a particular animal health condition may be calculated for each of these profiles. If only one animal profile defines the health condition and/or all of the profiles have been evaluated, the flow may continue to step 780. Otherwise, the flow may return to step 730 where the next animal profile may be evaluated.

At step 780, the health condition probabilities corresponding to each animal profile may be combined to calculate an overall heath-condition probability. This calculation may be made per Equation 1.13:

$${p}_{HC} = \frac{1}{N} {\sum }_{i = 1}^{N} {w}_{i} * {AP}_{HC , i} \qquad Eq . 1.13$$

In Equation 1.13, P_{HC} may represent an overall probability (e.g., combined over all animal profiles) that the animal has/is in a particular animal health condition (HC), N may represent the number of animal profiles having profile information relating to the particular health condition, AP_{HC.i} may represent the probability of the health condition according to a particular animal profile, i, and wᵢ may represent a proportional weight for the particular animal profile. For example, where an animal-specific profile, a group profile, and a breed/sex profile all comprise a health-condition pattern for a particular health condition (e.g., estrus), the overall probability of the health condition may be calculated per Eq. 1.14:

$${p}_{estrus} = \frac{1}{3} ( {w}_{estrus , AS} {Ap}_{estrus , AS} + {w}_{estrus , G} {Ap}_{estrus , G} + {w}_{estrus , B} {Ap}_{estrus , B} ) \qquad Eq . 1.14$$

In Equation 1.14 the number of animal profiles having an estrus health condition may be three, Ap_{estrus,AS} may represent the probability of estrus according to an animal-specific profile, Ap_{estrus,G} may represent the probability of estrus according to a group profile, and Ap_{estrus,B} may represent the probability of estrus according to a breed/sex profile. In Equation 1.14, the weights (e.g., w_{estrus,AS,} w_{estrus,G,} w_{estrus,B}), may represent proportional weights of each animal profile. As discussed above, these weights may be used where one of the profiles is considered to be a better indicator of an animal health condition than another. For example, a well-developed, animal-specific profile may be better at detecting a particular animal health condition than a breed/sex profile. Alternatively, a less-developed animal-specific, health-condition pattern may perform poorly relative to a better developed group profile. As such, weights w_{estrus,AS,} w_{estrus,G}, and w_{estrus.B} may be tailored the animal profile and/or performance of the detection of method 700.

At step 790, method 700 may determine whether additional animal health conditions may be evaluated. In one embodiment, all of the animal health conditions defined in a particular animal information data structure may be evaluated in method 700. For example, a probability that the animal is in a nominal health condition, an estrus health condition, and off-feed health condition may be calculated. If additional health conditions remain, the flow may continue to step 720 where the probability that the animal has/is in the next health condition may be calculated; otherwise, the flow may terminate.

Turning now to FIG. 8, a flow diagram of one embodiment of a method for detecting an animal health condition is depicted.

At step 810, overall probabilities that the animal has/is in a particular health condition may be received. These probabilities may have been calculated using method 700 discussed above in conjunction with FIG. 7.

At step 820, method 800 may iterate over all of the received health conditions. In this way, method 800 may be performed within loop 710-790 of FIG. 7 (which loops through all of the animal information data structure health conditions). Alternatively, method 800 may be preformed in a separate loop as depicted in FIG. 8.

At step 830, a probability of a particular animal health condition may be compared to a detection threshold for the health condition. The detection threshold may be stored in an animal information data structure described above in conjunction with FIG. 3a (e.g., as an entry in health condition settings 386). If the probability that the animal has/is in the health condition is greater than a detection threshold associated with the health condition, method 800 may continue to step 840; otherwise, the flow may continue to step 880.

At step 840, the time the probability has been maintained above (or near) the detection threshold of step 830 may be compared to a time threshold. The comparison of step 840 may require that the health condition probability of step 830 be maintained for a threshold period of time. As such, step 840 may be used to avoid erroneously detecting a health condition due to a short term “glitch” in animal behavior and/or measured animal characteristics. In some cases, the time period of step 840 may be very short and/or not used. For example, in detecting a health condition that has potentially serious, short-term consequences (e.g., the animal has gone off-feed and/or is out of the animal enclosure), the time threshold of step 840 may be very short and/or zero (0). If the probability has been maintained over the detection threshold for the time threshold, the flow may continue to step 850; otherwise, the flow may continue to step 880. Like the detection threshold, the time threshold may be maintained in an animal information data structure 310 of FIG. 3a (e.g., in health condition settings 386). These thresholds may be manually provided by an animal manager and/or may be pre-set.

At step 850, the health condition may be detected. Such detection may comprise determining that the animal has or is in the health condition and updating an animal information data structure (e.g., animal state 388 in FIG. 3a) accordingly.

At step 860, user preferences (e.g., health condition settings 386 of FIG. 3a) and/or other settings related to the detected health condition may be evaluated to determine whether the detection of the health condition warrants notifying and/or alerting an animal manager. As discussed above, some health conditions may require responsive action by an animal manager. Some health conditions (e.g., off-feed and/or out-of-enclosure) may be time sensitive. For example, an animal in estrus may be moved into a breeding pen, whereas an animal out of estrus (i.e., in a nominal state) may be removed from the pen. Since an animal estrus cycle may be relatively short, an animal manager may wish to be alerted as soon as a change in estrus condition is detected so as to be able to quickly respond to the change. Similarly, when an animal goes off-feed, an animal manager may wish to be informed since the condition may be indicative of a more serious health risk, and, if the animal is being raised for slaughter, any time off-feed may represent potential lost weight and subsequently, lost revenue. Rules for determining at 860 whether an animal manager should be alerted may be stored in an animal information data structure and/or settings related to the particular health condition (e.g., health condition settings 386 of animal information data structure 310 in FIG. 3a). If notification and/or alerting is required, the flow may continue to step 870. Otherwise, the flow may continue to step 880.

At step 870, a notification and/or alert relating to the detected animal health condition may be issued to an animal manager. An animal health condition alert may be made in a number of ways. For example, method 800 may be communicatively coupled to a communications network (e.g., as depicted in FIGS. 1 (network 115) and 4 (network 415)). As discussed above, the network may comprise any communications infrastructure and/or protocol known in the art. As such, the alert may comprise a network message identifying the health condition and the animal. Such a message may comprise an email, instant message (IM), short message service (SMS), or the like. Alternatively and/or in addition, Process 800 may be communicatively coupled to a public switched telephone network (PSTN) and/or cellular telephone network. As such, a voice message, pager message, text message, email, SMS, or the like may be dispatched using these alternative means. Additionally, the general and/or special purpose computing device performing method 800 may be communicatively coupled to an input/output (I/O) system comprising an audio speaker system and/or display. In these embodiments, the alert may comprise audible alert and/or visual alert. It should be understood that any combination alerting mechanisms known in the art could be used within the disclosed teachings and, as such, the disclosure should not be limited to any one or particular combination of alerting mechanisms. After dispatching the appropriate alert(s), the flow may continue to step 880.

At step 880, the current sensor settings, animal health condition, and/or health condition probabilities may be evaluated to determine whether the sensor settings should be modified. For instance, if the probability that a particular animal has/is in a particular health condition were to fall just under the detection threshold of step 830, step 880 may determine the animal should be monitored more frequently in order to quickly detect a possibly forthcoming change in animal health condition. Similarly, if the probability that an animal has a particular health condition is above the detection threshold at step 830, but the time threshold of step 840 was not met, and hence the health condition was not detected, step 880 may determine that monitoring should be done more frequently. In contrast, after increasing the monitoring frequency and detecting a change in animal health condition (e.g., from an estrus state to nominal), step 880 may reduce the monitoring frequency to reduce the power consumption of the animal sensors (e.g., bolus). As discussed above, the animal information data structure may comprise sensor settings (i.e., element 387 of FIG. 3a) which may reflect the current configuration state of the bolus and/or sensors. As such, step 880 may compare the current sensor settings (e.g., operational mode, polling frequency, sensor read duration, etc.) to required settings to determine whether a change is warranted. If the sensor settings are to be monitored, the flow may continue to step 885; otherwise, the flow may continue to step 890.

At step 885, a sensor settings modification message may be generated and/or updated. The sensor settings message may comprise changes to sensor settings identified at step 885 (e.g., increasing and/or decreasing monitoring frequency of one or more animal characteristics). In addition, the operational mode of the sensor device (e.g., bolus) may be updated. As discussed above, a bolus may operate in “burst” and/or “instantaneous mode.” The message of step 885 may change the operational mode of bolus in order to conserve power or monitor a particular animal characteristic more closely. Similarly, the message of step 885 may activate and/or deactivate one or more bolus sensors, increase and/or decrease the sampling and polling frequency of one or more sensors, or the like. After generating the message, sensor settings stored in the animal information data structure may be updated, and the flow may continue to step 890.

At step 890, method 800 may determine whether there are additional animal health condition probabilities to process. If so, the flow may continue to step 820 where the next animal health condition probability may be evaluated; otherwise, the flow may continue to step 895.

At 895, an animal information data structure associated with the animal may be updated to reflect any detected animal-health conditions, the probabilities received at step 810, timing information relating to step 840, or the like. Such information may be used as the animal is monitored on an on-going basis (e.g., as subsequent animal characteristics messages are received). For example, information that a probability of a particular health condition was over the detection threshold but under the time threshold, may be used on a subsequent iteration of method 800 to establish a time threshold requirement of the health condition at step 840. In addition, at step 895, if a sensor settings message was created at step 885, it may be transmitted. Such transmission may be performed via a base station in wireless communication with the sensor (e.g., bolus). As discussed above, the base station may be communicatively coupled to method 800.

It will be obvious to those having skill in the art that many changes may be made to the details of the above-described embodiments without departing from the underlying principles of the invention. The scope of the present invention should, therefore, be determined only by the following claims.

I claim:

1. A method for detecting a health condition of an animal, comprising:
receiving a message from an animal monitoring device, the message comprising a measured animal characteristic;
comparing the measured animal characteristic to an animal characteristic model corresponding to an animal health condition;
calculating a probability that the animal has the health condition based upon the comparing; and
detecting the health condition if the probability is above a detection threshold.

2. The method of claim 1, wherein the animal monitoring device is an ingestible bolus comprising a sensor disposed therein.

3. The method of claim 2, further comprising updating a bolus setting responsive to the comparing.

4. The method of claim 2, wherein the animal is a ruminant animal, and wherein the ingestible bolus is disposed within a stomach of the animal.

5. The method of claim 2, wherein the sensor is an accelerometer.

6. The method of claim 5, wherein the accelerometer is a three-axis accelerometer and the measured animal characteristic is a derivative of a vector magnitude of animal acceleration.

7. The method of claim 6, wherein the measured animal characteristic is an animal stomach contraction measured by the accelerometer.

8. The method of claim 1, wherein detecting the health condition comprises determining whether the probability has been maintained above the detection threshold for a time threshold, and wherein the health condition is detected if the time threshold determining is positive.

9. The method of claim 1, wherein the message comprises a plurality of measured animal characteristics, and wherein calculating a probability that the animal has the health condition comprises combining a plurality of probabilities based on comparing each of the plurality of measured animal characteristics corresponds to one of a plurality of animal characteristic models associated with the animal health condition.

10. The method of claim 9, wherein the combining comprises applying a separately defined proportional weight to each of the plurality of probabilities.

11. The method of claim 1, wherein the animal characteristic model is common to a group of animals.

12. The method of claim 1, wherein the animal characteristic model is common to an animal breed.

13. The method of claim 1, wherein comparing the measured animal characteristic to an animal characteristic model associated with the health condition comprises making a first comparison of the measured animal characteristic to an animal-specific animal characteristic model and making a second comparison of the measured animal characteristic to an animal characteristic model common to a group of animals, and wherein calculating a probability based on the comparing comprises combining a probability based on the first comparison and a probability based on the second comparison.

14. The method of claim 13, wherein calculating comprises applying a first proportional weight to the probability based on the first comparison and a second proportional weight to the probability based on the second comparison.

15. The method of claim 1, wherein the animal characteristic model is a statistical model.

16. The method of claim 15, wherein the model is a normal distribution of the animal characteristic defined by a mean and a standard deviation.

17. The method of claim 1, wherein the health condition is one selected from the group consisting of a nominal condition, an off-feed condition, a colic condition, an out-of-enclosure condition, and an estrus condition.

18. A computer readable storage media comprising computer readable instructions to perform a method, the method comprising:
receiving an animal characteristics message from a sensor disposed within a stomach of a ruminant animal, the message comprising an animal characteristic measured by the sensor;
comparing the measured animal characteristic to a statistical animal characteristic model of an animal health-condition pattern;
calculating a probability that the animal has the health condition based upon the comparing; and
detecting the health condition if the probability is above a detection threshold for a time threshold.

19. The computer readable storage media of claim 18, wherein the method further comprises alerting an animal manager of the detected health condition if the probability is above the detection threshold for the time threshold.

20. The computer readable storage media of claim 18, wherein the method further comprises modifying a setting of the sensor based on the comparing.

21. The computer readable storage media of claim 18, wherein the animal characteristics message comprises a plurality of measured animal characteristics, and wherein the method further comprises,
comparing each of the plurality of measured animal characteristics to a statistical animal characteristic model corresponding to the animal health-condition, and
calculating the probability that the animal has the health condition by combining each of the probabilities based on each of the comparisons.

22. The computer readable storage media of claim 18, wherein comparing the measured animal characteristic to a statistical animal characteristic model associated with the health-condition pattern comprises:
performing a first comparison between the measured animal characteristic and a first statistical animal characteristic model common to a group of animals,
performing a second comparison between the measured animal characteristic and a second statistical model common to an animal breed, and
wherein the probability that the animal has the health condition is based on a combination of the first comparison and the second comparison.

23. A method for detecting a health condition of an animal, the method comprising:
receiving a message from a animal monitoring device disposed within a stomach of a ruminant animal, the message comprising an animal characteristic measured by a sensor disposed within the animal monitoring device;
comparing the measured animal characteristic to a first statistical model associated with a health condition, wherein the first statistical model is common to a group of animals;
comparing the measured animal characteristic to a second statistical model associated with the health condition, wherein the second statistical model is common to an animal breed;
calculating a heath-condition probability that the animal has the health condition based upon the first comparing and the second comparing, wherein the calculating comprises combining a first probability based on the first comparing with a second probability based on the second comparing; and
detecting the health condition if the health-condition probability is above a detection threshold.

24. A system for monitoring a health condition of an animal, comprising:
an ingestible bolus disposed within a stomach of a ruminant animal, the bolus comprising a sensor, a power source, and a wireless communication module;
a base station in wireless communication with the ingestible bolus; and
a computing system communicatively coupled to the base station, the computer system comprising:
a communication interface configured to receive an animal characteristics message comprising a measured animal characteristic from the bolus,
a data storage module to store an animal information data structure associated with the animal,
a heath-condition monitor configured to compare the measured animal characteristic to a statistical model of the characteristic, wherein the statistical model is indicative of an animal health condition, and wherein the health-condition monitor is configured to calculate a probability that the animal has a health condition based on the comparison, and wherein the health-condition monitor is configured to detect the health condition if the probability is above a detection threshold for a threshold time period.

25. The system of claim 24, wherein the computing system is communicatively coupled to a communication network, the computing system further comprising an alerting module to issue and alert to an animal manager using the communication network responsive to detecting the health condition.
